(19) United States
(12) Patent Application Publication
(10) Pub. No.: US 2017/0165427 A1
(43) Pub. Date: Jun. 15, 2017

(54) SYRINGE AND FLUID INJECTION SYSTEM WITH AN ORIENTATION INDEPENDENT IDENTIFICATION CODE

(71) Applicant: BAYER HEALTHCARE LLC, WHIPPANY, NJ (US)

(72) Inventors: ARTHUR E. UBER, III, PITTSBURGH, PA (US); EDWARD K. PREM, ALLISON PARK, PA (US); HOWELL TIMOTHY GOLDREIN, CAMBRIDGE (GB)

(73) Assignee: BAYER HEALTHCARE LLC, Whippany, NJ (US)

(21) Appl. No.: 15/325,138

(22) PCT Filed: Jul. 14, 2015
(86) PCT No.: PCT/US2015/040305

§ 371 Date: Jan. 10, 2017

Related U.S. Application Data
(60) Provisional application No. 62/024,216, filed on Jul. 14, 2014.

(51) Int. Cl. A61M 5/31; A61M 5/00; A61M 5/168; A61M 5/145

(52) U.S. Cl. CPC A61M 5/3135; A61M 5/14546; A61M 5/007; A61M 5/16804; A61M 2205/60; A61M 2205/6072; A61M 2205/18

(57) ABSTRACT

A syringe is provided including an orientation independent indicia that can be used to validate and determine information about the syringe, injection parameters, and/or fluid contained therein. The syringe includes a syringe barrel and an indicia located on a portion of the syringe containing or associated with identifying information about the syringe, one or more injection parameters of a fluid injector, or a fluid filled in the syringe barrel. The indicia is readable by a reader independent of an orientation of the syringe relative to the reader. A fluid injection system for providing fluid into a patient is also provided. The injection system includes the syringe having an orientation-independent indicia, an injector having at least one syringe receiving port for receiving the syringe, and a controller. The controller can be configured to obtain information from the indicia and provide instructions for performing injections to the injector, based on the information.

### CROSS REFERENCE TO RELATED APPLICATION

The present application claims priority to U.S. Provisional Application No. 62/024,216 filed Jul. 14, 2014, the disclosure of which is incorporated by reference herein.

### BACKGROUND

Field of the Technology

The present disclosure relates generally to a syringe for containing a fluid for injection into a patient and, more specifically, to a syringe with an orientation independent indicia that can be used to validate and determine information about the syringe, injection parameters, and/or fluid contained therein.

Description of Related Art

In many medical, diagnostic, and therapeutic procedures, a medical practitioner, such as a physician, injects a patient with a medical fluid. In recent years, a number of injector-actuated syringes and powered injectors for pressurized injection of fluids, such as contrast media (often referred to simply as “contrast”), medicaments, or saline, have been developed for use in imaging procedures such as angiography, computed tomography, ultrasound, and magnetic resonance imaging. In general, these powered injectors are designed to deliver a preset amount of contrast or other fluid at a preset flow rate.

For example, angiography is used in the detection and treatment of abnormalities or restrictions in blood vessels. In an angiographic procedure, a radiographic image of a vascular structure is obtained through the use of a radiographic contrast, which is injected through a catheter. The vascular structures, in fluid connection with the vein or artery into which the contrast is injected, are filled with contrast. X-rays passing through the region of interest are absorbed by the contrast, causing a radiographic outline or image of blood vessels containing the contrast. The resulting images can be displayed on, for example, a video monitor and recorded.

In a typical imaging procedure involving the use of a contrast media, the medical practitioner places a cardiac catheter into a vein or artery. The catheter is connected to either a manual or an automatic contrast injection mechanism. A typical manual contrast injection mechanism includes a syringe in fluid connection with a catheter connection. The fluid path also includes, for example, a source of contrast and a source of flushing fluid, typically saline. The operator of the manual contrast injection mechanism controls the syringe to draw saline or contrast into the syringe and to inject the contrast or saline into the patient through the catheter connection. Automatic contrast injection mechanisms typically include at least one syringe connected to a fluid injector and, for example, driven by at least one linear actuator of the injector. The linear actuator operates a piston rod configured to contact and engage a moveable plunger of the syringe. The contrast and saline are drawn into the at least one syringe by withdrawing the plunger in a proximal direction through a barrel of the syringe. The contrast and saline are then selectively injected to the patient via the catheter connection according to an injection protocol or injection parameters.

It is often necessary for the operator or technician to validate the syringe prior to performing the injection. Validation can include confirming that the syringe is acceptable for the injector or injection procedure and determining various characteristics of the syringe and fluid contained or to be contained therein. Validation may also include confirming that the syringe assembly is genuine (e.g. to prevent counterfeiting, use of inferior or miss-fitting syringes that may result in improper mating between the syringe assembly and injector or may not have the required tolerances for a particular injection procedure, possibly resulting in malfunction during the procedure). For example, the operator must verify that identifying information, such as the syringe dimensions (e.g., diameter, length, and fluid volume), and fluid contents are correct for the procedure being performed. In addition, the operator can be required to provide certain information about the syringe, such as the date of manufacture, source, frictional characteristics between the plunger and syringe barrel, fluid viscosity, and the like (referred to generally hereinafter as “injection parameters”) to the fluid injector or the injector operating system to control piston force and acceleration to deliver fluid at a desired and controlled flow rate. Other important identifying information may include manufacturer, lot number, expiration date or shelf-life indicator, etc. Including the various desired identifying information may be difficult given the amount of available surface area and aesthetic reasons. For example, if the identifying information covers too much surface area on the syringe, it may impact the technician's ability to determine if the syringe has been properly filled. The identifying information can be printed on a product label attached to the syringe packaging or body. This information can also be printed directly on the syringe body. In some systems, the fluid injector can include a sensor or reader located on the injector itself for automatically reading the label or tag when the syringe is inserted in the injector. The fluid injector uses the information extracted from the label or tag to validate the syringe and to control the injection.

Generally, an injector includes at least one syringe insertion port configured to receive a proximal end of the corresponding syringe. The syringe insertion port can require that the syringe is inserted in a correct position or orientation for connection with the linear actuator mechanism. In that case, the sensor or reader can be positioned to identify and read a tag located on a corresponding portion of the syringe body. However, some fluid injectors can accept a syringe in multiple orientations or in any orientation. In that case, the operator or technician must rotate the syringe until the sensor or reader is properly aligned to the tag or label. This step of rotating the syringe to correctly align the sensor and tag increases time required to prepare the injector for an injection and can result in inaccuracies during the injection procedure.

### SUMMARY

In view of the above difficulties, there is a need for an improved optical code or tag that can be applied to a syringe barrel and/or piston. The code or tag should be adequate to provide enough identifying information about the syringe and injection parameters to permit automatic syringe validation and injection control without requiring additional review, input or data entry by the technician or operator. Furthermore, the tag or code should be sufficiently direction-independent that the technician is not required to rotate the syringe to align the tag or code with the sensor or reader. The syringe and fluid injection system described herein addresses these and other issues.

According to an aspect of the disclosure, a syringe includes: a syringe barrel having a proximal end, a distal end, and a sidewall extending therebetween; and at least one indicia located on a portion of the syringe containing or associated with identifying information about the syringe, one or more injection parameters of a fluid injector, or a fluid filled in the syringe barrel. The indicia is readable by a reader independent of an orientation of the syringe relative to the reader.

In some examples, the indicia includes one or more markings etched into a portion of the syringe barrel. The indicia can also include one or more computer readable barcodes. For example, the indicia can include a plurality of repeating barcode sections. Each repeating bar code section can include all of the identifying information of the indicia.

In some examples, each of the plurality of barcode sections includes straight markers, indicating lines on the barcode section that should be identified as straight when processing the barcode.

In some examples, the indicia is located on a portion of the syringe barrel configured to be inserted into a syringe port of a fluid injector. The indicia can also be located on a proximal surface or a distal surface of an annular flange extending from the syringe barrel.

In some examples, the at least one indicia comprises a first set of parallel lines at least partially overlapping with a second set of parallel lines at a different orientation from the first set of parallel lines, thereby forming a moiré pattern. The first set of parallel lines and the second set of parallel lines can be each located on the syringe barrel. The syringe can include a plunger and a piston rod. The second set of parallel lines can be located on at least one of the piston rod and the plunger.

According to another aspect of the disclosure, a fluid injection system is provided. The system includes at least one syringe with a syringe barrel having a proximal end, a distal end, and a sidewall extending therebetween; at least one indicia on at least a portion of the syringe barrel; a fluid injector having at least one syringe receiving port configured to receive the at least one syringe; at least one sensor positioned to obtain an image of the at least one indicia; and a controller. The controller can be configure to: obtain the image from the at least one sensor; read the image to extract identifying information about the at least one syringe, one or more injection parameters, or information for a fluid contained or to be contained in the at least one syringe; and provide instructions for performing an injection using the fluid injector, the instructions being based, at least in part, on the information extracted by the controller. The at least one indicia can be orientation independent, and can be read by the sensor in any position relative to the syringe receiving port.

In some examples, the at least one indicia includes a plurality of barcode sections arranged around a circumference of the syringe barrel. Each of the plurality of barcode sections can include four or more location identifying markers. In that case, the sensor can include a field of view having an area large enough to capture an image including four or more markers from either one barcode section or from an adjacent barcode section, regardless of the orientation of the syringe relative to the injector port. For example, a side length of the field of view of the sensor can be at least \sqrt{2} times the length of a side length of one of the plurality of barcode sections.

In some examples, reading the image of the at least one indicia can include applying an image processing algorithm to translate one or more portions of the image to produce a correctly oriented barcode section. In other examples, reading the image of the at least one indicia can include applying an image processing algorithm to account for curvature of the syringe barrel. Further, each barcode section can include straight markers, indicating portions of the barcode that should be read as straight lines regardless of the orientation of the line in the image captured by the sensor.

In some examples, the at least one indicia can include a first set of parallel lines at least partially overlapping with a second set of parallel lines at a different orientation from the first set to form a moiré pattern. The first set of parallel lines can be located on the syringe barrel, and the second set of parallel lines can be located on at least one of a piston rod of the fluid injector and a plunger of the at least one syringe.

In some examples, reading the image of the at least one indicia includes identifying a spatial relationship between the first set of parallel lines and the second set of parallel lines. In addition, the controller can be configured to identify an injection rate based on the spatial relationship between the first set of parallel lines and the second set of parallel lines.

### BRIEF DESCRIPTION OF THE DRAWINGS

Some of the advantages and features of the various embodiments of the disclosure have been summarized herein. These embodiments will become apparent to those skilled in the art when referencing the following drawings in conjunction with the detailed descriptions as they relate to the figures.

FIG. 1 is a schematic view of a system including a powered injector and at least one syringe according to an embodiment of the disclosure;

FIG. 2 is a front view of an embodiment of a syringe for use with the injector of FIG. 1, according to an embodiment of the disclosure;

FIG. 3A is a schematic view of an orientation independent barcode and reader window according to an embodiment of the disclosure;

FIG. 3B is a schematic view of the orientation independent barcode and reader window of FIG. 3A with the reader window in a different orientation relative to the barcode;

FIG. 4A is a front view of a syringe having an identification tag formed from a moiré pattern according to an embodiment of the disclosure;

FIG. 4B is an enlarged view of the proximal end of the syringe of FIG. 4A;

FIG. 5 is a schematic view of an embodiment of a moiré pattern;

FIG. 6 is a schematic view of another embodiment of a moiré pattern;

FIG. 7A is a side view of a syringe and piston rod according to another embodiment of the disclosure; and

FIG. 7B is a front view of the piston rod of FIG. 7A inserted in the syringe barrel.

### DETAILED DESCRIPTION

For purposes of the description hereinafter, the terms “upper”, “lower”, “right”, “left”, “vertical”, “horizontal”, “top”, “bottom”, “lateral”, “longitudinal”, and derivatives thereof shall relate to the disclosure as it is oriented in the drawing figures. When used in relation to the syringe, the term “proximal” refers to the portion of the syringe nearest the injector, when the syringe is connected to the injector. The term “distal” refers to the portion of the syringe farthest away from the injector. It is to be understood, however, that the disclosure can assume alternative variations and step sequences, except where expressly specified to the contrary. It is also to be understood that the specific devices and processes illustrated in the attached drawings, and described in the following specification, are simply exemplary embodiments of the disclosure. Hence, specific dimensions and other physical characteristics related to the embodiments disclosed herein are not to be considered as limiting.

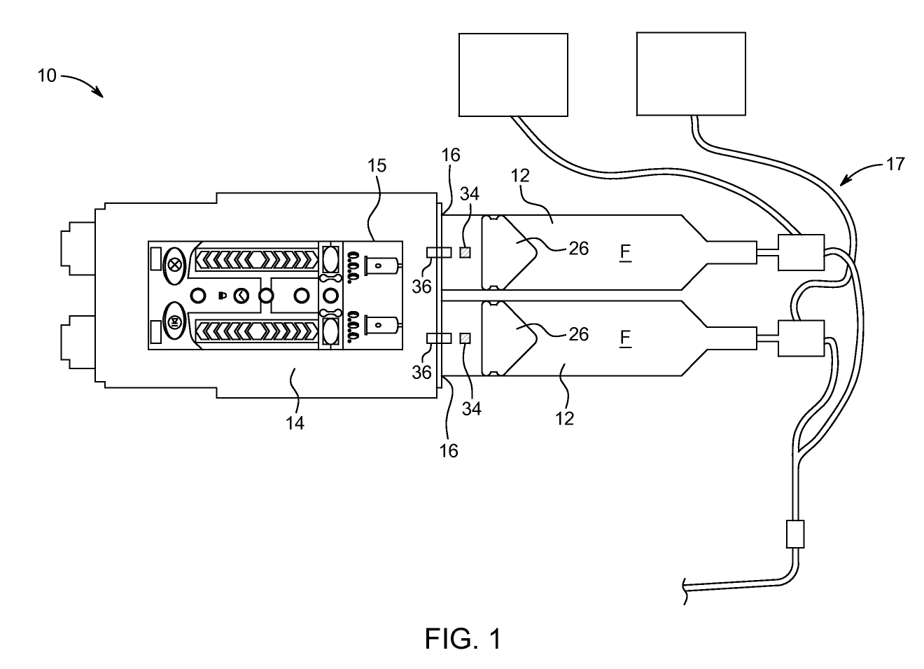

With reference to FIG. 1, an injector 10, such as an automated or powered fluid injector, is illustrated, which is adapted to interface with and actuate one or more syringes 12, which can be filed with a medical fluid F, such as contrast media, saline solution, or any desired medical fluid. The injector 10 can be used during an angiographic, computed tomography (CT), magnetic residence imaging (MRI), molecular imaging, or other medical procedure to inject contrast and common flushing agents, such as saline, into the body of a patient prior to, during, and/or after an imaging procedure. According to certain embodiments, the injector 10 can be at least a dual-syringe injector, wherein the two fluid delivery syringes 12 are oriented in a side-by-side or other spatial relationship and are separately actuated by respective linear actuators or piston elements of the injector 10. Other powered injectors may include a single syringe or three or more syringes.

The injector 10 can be enclosed within a housing 14 formed from a suitable structural material, such as plastic and/or metal. The housing 14 can be in various shapes and sizes depending on the desired application. For example, the injector 10 can be a free-standing structure configured to be placed on the floor or can be a smaller design for placement on a suitable table or support frame. The injector 10 includes one or more syringe ports 16 for reversibly interfacing with syringes 12 and for connecting the proximal end of a plunger 26 of the syringe 12 to piston elements of the injector 12. The at least one syringe port 16 can be located on a side of the housing 14. The housing 14 can be rotatable to direct the distal end or nozzle of the syringe 12 in a vertical, horizontal, or downward facing direction.

The syringe 12 includes at least one indicia or identification tag or strip 34 positioned on at least a portion of the syringe 12, such as at least partially around the outer circumference at a proximal portion of the syringe 12. The at least one indicia or identification tag or strip 34 can be embedded or otherwise associated with identifying characteristics or other identifying information about the syringe 12, including one or more of the syringe type, physical dimensions, flow characteristics, fluid contents, fluid source or type, manufacturer of the syringe, lot number, date of manufacture of the syringe, expiration of use date (i.e., the maximum shelf-life of the syringe), and similar syringe information, as described herein. In some examples having more than one identification tag or strip 34, each identification tag 34 can contain at least a portion of the total information embedded within or otherwise associated with the identification tag 34. In other examples, each identification tag 34 can contain specific syringe information or injection parameters. The at least one identification tag 34 can be read by at least one sensor 36, positioned on or recessed in the side of the housing 14 or within at least a portion of the inner surface of the at least one syringe port 16 of the injector 10.

With continued reference to FIG. 1, the injector 10 includes at least one fluid path set 17 interfaced with a distal port or nozzle of the syringe 12 for delivering one or more fluids from each of the at least one syringes 12 to a catheter, needle, or other inserted fluid path (not shown) inserted into a patient at a vascular access site. For example, a flow of saline solution from a first syringe 12 and contrast from a second syringe 12 can be regulated by a fluid control module (not shown) associated with the injector during an injection procedure. The fluid control module is configured to operate at a variety of injection rates and pressures to regulate the delivery of the saline solution and/or contrast to the patient, based on user selected injection parameters, such as injection flow rate, duration, total injection volume, and ratio of contrast media and saline, which can be programmed or otherwise entered into the injector fluid control module. An exemplary front-loading fluid injector is disclosed in U.S. Pat. No. 5,383,858, which is incorporated by reference in its entirety. Other multi-fluid delivery systems are disclosed in U.S. Pat. No. 7,553,294; U.S. Pat. No. 7,666,169; International Patent Application Publication No. WO 2012/155035; and United States Patent Application Publication No. 2014/0027009; all of which are assigned to the assignee of the present application, and the disclosures of which are incorporated herein by reference. The principles of this disclosure may be applied to fluid injectors that operate only a single syringe assembly 12 assembly or multiple syringe assemblies, such as, for example two or three syringe assemblies.

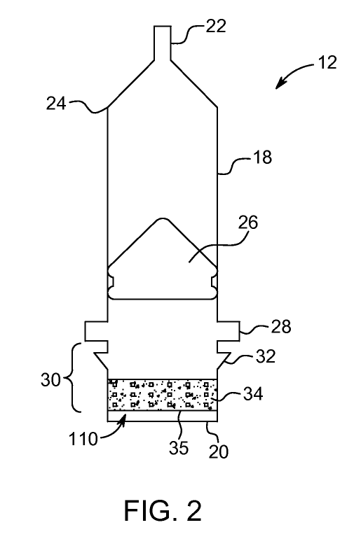

Having described the general structure and function of the injector 10, the syringe 12, which is configured to contain a medical fluid F, will now by discussed in detail. With reference to FIG. 2, in some examples, the syringe 12 includes a generally cylindrical barrel 18 formed from glass or a suitable medical-grade plastic. The barrel 18 includes the proximal end 20 and the distal end 24, with a nozzle 22, extending therefrom. The proximal end 20 of the syringe barrel 18 can be sealed with the plunger 26, such as an elastomeric plunger, slidably disposed within the barrel 18. The plunger 26 forms a liquid tight seal against the sidewall of the barrel 18 as it is advanced or withdrawn therethrough through the action of a piston actuator associated with injector 10.

In some examples, the syringe 12 also includes an annular flange (referred to as a drip flange 28) extending from the outer surface of the barrel 18 at a position near the proximal end 20 thereof. When the syringe 12 is inserted in the injector 10 (shown in FIG. 1), the drip flange 28 may rest against a distal opening of the syringe port 16 (shown in FIG. 1) to prevent fluid expelled from the syringe 12 from entering the port 16 and fouling the interior workings of injector 10. The portion of the syringe barrel 18 near the proximal end 20, for example between the drip flange 28 and the proximal end 20 of the syringe 12, referred to hereinafter as the insertion portion 30 of the syringe barrel 18, is sized and adapted to be inserted in the syringe port 16 of the injector 10. Accordingly, in certain examples, the insertion portion 30 of the barrel 18 includes one or more locking structures, such as a locking flange 32, extending outward from the barrel 18. The locking flange 32 or other locking structure can be adapted to form a locking engagement with corresponding protrusions or locking structures within the syringe port 16 of the injector 10 for releasably holding the syringe 12 in the syringe port 16 while the injector 10 is in use. Alternatively, the insertion portion 30 can include various latches, locking mechanisms, or radially extending ribs for connection to corresponding portions of the syringe port 16. Examples of syringes that can be used with the injector 10 of FIG. 1 are described in U.S. Pat. No. 5,383,858, the disclosure of which is incorporated herein by reference in its entirety. Other exemplary syringes are described in U.S. Pat. No. 6,322,535 and U.S. Pat. No. 6,652,489, the disclosures of which are both incorporated herein by reference.

With continued reference to FIG. 2, in some examples the syringe 12 includes the at least one indicia or identification tag or strip 34 positioned near and surrounding at least a portion of an outer circumference of the proximal end 20 of the syringe barrel 18. In other examples, the at least one indicia or identification tag 34 can be located on a portion of the drip flange 28 or locking flange 32, for example, on the underside (e.g., the proximal facing side) of the drip flange 28, the topside (e.g., the distal facing side) of the drip flange 28, or in another convenient location. The at least one indicia or identification tag 34 can be a position and/or orientation independent barcode 110. As defined herein, orientation independent refers to an indicator tag 34 that can be read manually or automatically by a sensor regardless of the precise position of the indicator tag 34 relative to the user's eye or sensing field of the sensor. For example, an orientation independent bar code 110 can be read regardless of the position of the barcode 110 relative to the injector 10 or injector port 16.

In some examples, the barcode 110 can extend around the entire circumference of syringe barrel 18 providing relative rotational and lateral position independence and/or tolerance for reading or imaging by the one or more sensors. In other examples, the barcode 110 can be located at certain specific portions of the circumference of the syringe barrel 18 and the injector port 16 may be designed so that the syringe self-orients itself into a position where the barcode 110 may be imaged by one of the one or more sensors. In some examples, the orientation independent barcode 110 can be printed on a label 35 that is affixed at least partially around the circumference of the syringe barrel 18. Alternatively according to other embodiments, the barcode 110 can be printed directly on the barrel 18 using a printing technology for printing on plastic or glass curved surfaces. In another example, the barcode 110 can be formed on or within the surface of the barrel 18, for example, by etching, laser marking, heat forming, branding, or molding on the barrel 18 surface during manufacturing.

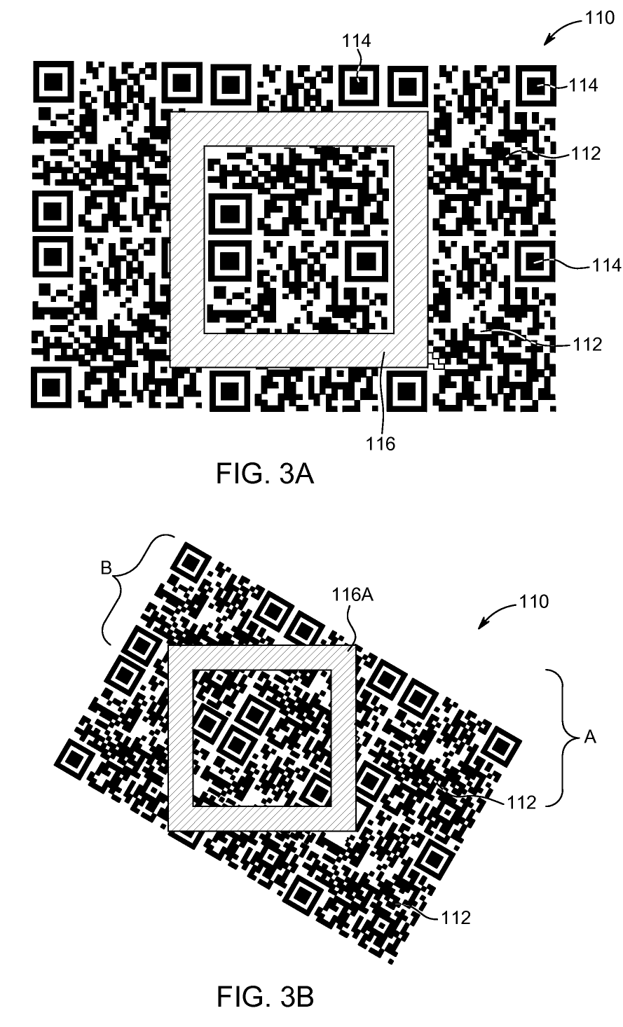

With reference to FIGS. 3A and 3B, one example of a position and/or orientation independent barcode 110 can include a field of at least two identical two-dimensional barcode sections 112 arranged next to one another in a repeating pattern. A two-dimensional barcode 110 can include a geometric pattern containing light and dark sections, which can be read by at least one sensor, for example, in the form of an imaging device, such as a digital camera, a CCD reader, omni-directional scanner, laser scanner or other imaging device. Certain two-dimensional barcodes are also referred to as Quick Read (QR) codes. An alternative suitable barcode is an Aztec code. According to one embodiment, each section 112 of a QR code can include at least three section markers 114 (including position markers and alignment markers, indicated as outlined black squares) positioned adjacent to the corners of the section 112. The barcode 110 is intended to be read using at least one sensor, such as a scanner or reader having a rectangular or square scan window 116 or field of view. As shown in FIG. 3A, the scan window 116 is large enough to encompass at least a full field of data (e.g., an entire barcode section 112), including all of the section markers 114, although the orientation of the markers 114 within the scan window 116 may change based on the relative position of the scan window 116 on the portion of the barcode 110 being read. By recognizing the markers 114 and positioning by translation of the associated region of code, a normal QR code can be created that can be then read by normal algorithms. Therefore, no matter the position of the scan window 116, the at least one sensor is able to read and extract all information from an entire barcode section 112.

With specific reference to FIG. 3B, the dimensions of the scan window 116a can also be increased if rotational misalignment between the scan window 116a and barcode 110 is an issue. For example, the scan window 116a can be at an angle relative to the barcode 110. According to this example, a scan window 116a having a side length A that is at least \sqrt{2} times the length of the section side length B is large enough to encompass an entire section 112 regardless of the orientation of the scan window 116a relative to barcode 110.

Once an image of the portion of the barcode 110 within the scan window 116 is captured, rotational and translation image processing can be performed to extract embedded information from the barcode 110. The image processing can include automatically reorienting the captured portions of the barcode 110 prior to reading the data therefrom. The data can be read using processing algorithms, including, for example, Reed-Solomon error correction in which the required data may be extracted from the barcode, even if a portion of the barcode has been damaged. In some examples, there can be some distortion in the extracted image due to the curvature of the surface of the syringe barrel 18 (shown in FIG. 2). The curvature can be accounted for optically through selection of an appropriate lens for the at least one image capture sensor or reader. Alternatively, the curvature can be accounted for digitally using an image capture sensor having sufficient pixel resolution and one or more algorithms for addressing such distortions. The curvature can also be addressed by adjusting features of barcode 110. For example, the predefined straight edges of the data grid and the markers 114 of a two-dimensional bar code can be adapted to facilitate this compensation for distortion due to curvature and/or for positional misalignment.

With reference again to FIG. 2, the at least one indicia or identification tag 34 can be embedded or otherwise associated with information, such as identifying information described herein, about the syringe 12, the injection parameters, and/or fluid contained therein. Alternatively, in certain embodiments, relevant information is not embedded on the tag 34 itself, but the identification tag 34 can contain information that serves as a pointer directing the at least one sensor, computer, injector, information system, and/or controlling device to an information storage location, such as on a computer database, computer network, internet, and/or similar data storage location, where specific information about the syringe 12, injection parameters, and/or syringe contents may be located. In other embodiments, the at least one indicia or identification tag 34 may include a portion of embedded information and a portion that serves as a pointer to an information storage location. The information regarding the information storage location can be read by at least one sensor 36 on the injector 10. The stored information can then be downloaded to a computer accessory viewable by the injector operator and/or directly to the injector or injector operating system, for preparing, adjusting, and/or modifying injection parameters. For example, the information can include the physical dimensions of the syringe 12 (e.g., length and width, fluid volume, nozzle dimensions, etc.). In addition, the at least one identification tag 34 can provide injection parameter information for the syringe 12, including the date of manufacture, lot number and/or source, expiration of use date of the at least one syringe barrel/plunger friction characteristics, pressure limitations, maximum or minimum flow rates, and/or fluid type. The physical parameter information and injection parameter information can be used to determine a preferred injection force, injection velocity, and/or an appropriate power level for the linear actuator of the injector 10. The information can also be used to determine if the syringe is appropriate for the indicated usage (i.e., wrong size, expired life-term, possible defects). Examples of types of relevant information that can be communicated and/or transmitted between a syringe and an injector and/or operating system is discussed, for example, in U.S. Pat. No. 6,743,202, the disclosure of which is incorporated herein by reference in its entirety. Further examples of types of information that can be communicated or transmitted between a syringe and an injector, and which could be stored on the identification tag 34 are disclosed in U.S. Pat. No. 5,739,508, the disclosure of which is incorporated herein by reference in its entirety.

With reference again to FIG. 1, in use, an operator inserts the proximal end 20 of the at least one syringe 12 into the at least one syringe port 16. The operator can be required to exert some force against each syringe 12 so that the feature, such as locking flange 32 or other locking lug assembly of the syringe 12 engages with corresponding locking structures (not shown) of the syringe port 16 to form a suitable reversible connection therewith. In some examples, the operator continues to press the syringe 12 into the port 16 until the insertion portion 30 of the syringe barrel 18 is entirely inserted. In some cases, an audible signal, such as a click, indicates that the syringe barrel is fully inserted, locked, and ready for use. Certain syringe locking features may not require orientation, such as an orientation free system or a self-orienting locking system.

Once the at least one syringe 12 is inserted in the at least one port 16, the at least one sensor 36 captures an image of at least a portion of the tag 34, for example the orientation independent barcode 110 or other tag or feature described herein. A controller 15 and/or processor in communication with the injector 10 and the at least one sensor 36 may then process the image to extract the information therefrom. The extracted information is used to identify the syringe 12, injection fluid F, and injection parameters, as described herein. This information can be used by the injector 10 and/or technician to prepare, modify or adapt the injector settings prior to initiating the injection process. Similarly, the injector 10 can be configured to provide an alarm or warning and/or cancel the injection procedure if the loaded at least one syringe 12 and/or fluid F contained therein is inappropriate for the procedure to be performed, optionally accompanied by a visual or audible warning to alert the technician to the existence of the problem. Assuming that the at least one syringe 12 and/or fluid F are appropriate for the procedure, the injector 10 can automatically begin the injection procedure or provide an alert to the technician that the syringe is validated and that the injection procedure may be initiated. In the latter case, the operator can be required to initiate the injection by an actuation activity, such as pressing a start button. Once the injection is actuated, the linear actuator of the injector 10 contacts and engages the proximal end of the plunger 26 disposed within the syringe barrel 18. Movement of the at least one plunger 26 in the proximal direction draws fluid F into the at least one syringe 12, for example when the at least one syringe is not a pre-filled syringe. Movement in the distal direction expels fluid F contained within the at least one syringe 12, thereby injecting fluid F into the patient through any known injection structure, such as an IV tube or needle accessory.

According to other embodiments, the at least one indicia or marking 34 on syringe 12 may include at least one moiré pattern. To generalize, a moiré pattern can be generated by the interference of at least two visually overlapped sets of substantially parallel lines or other repeating markings that can differ slightly in spacing (translational frequency) and/or angle (tilt) to create a unique pattern that may be associated with particular information.

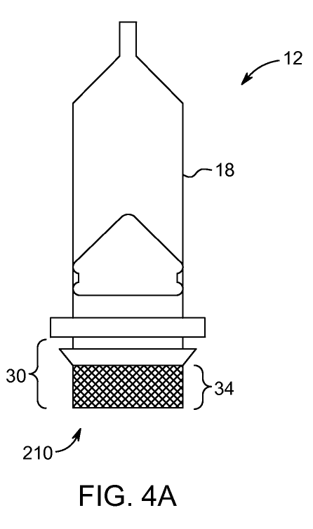

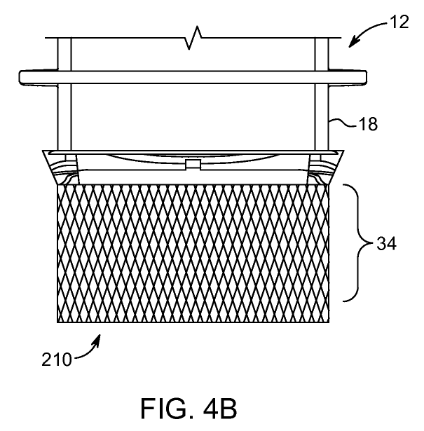

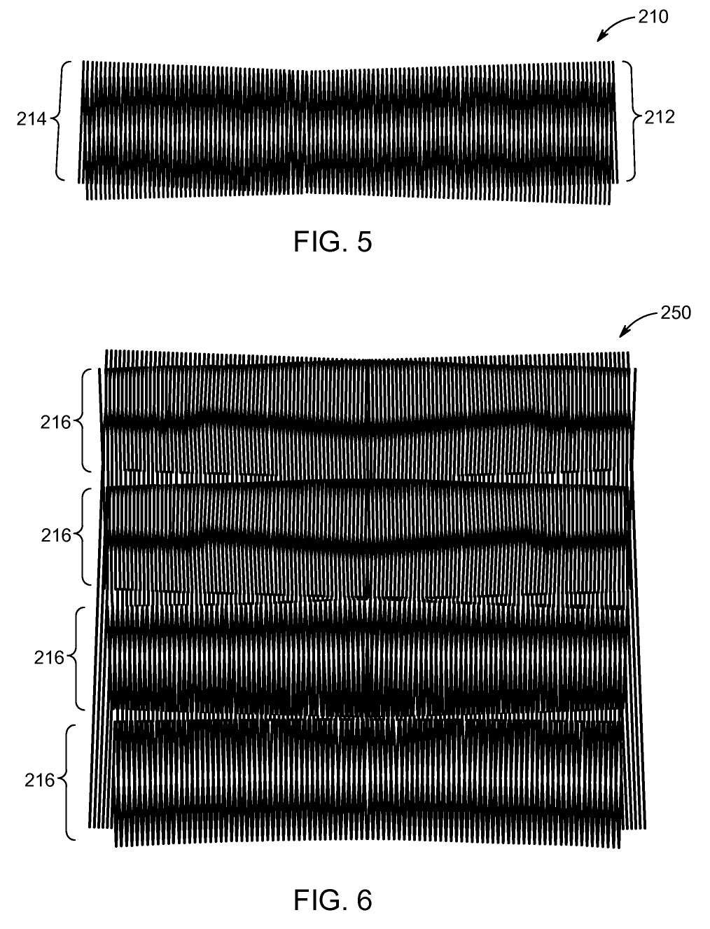

With reference to FIGS. 4A and 4B, the at least one syringe 12 can include an orientation independent indicia or identification tag 34 formed from a series of overlapping lines arranged to create a moiré pattern 210. For example, the moiré pattern 210 may be formed from a set of fine parallel lines overlapped by at least one additional set of fine parallel lines that are angled or otherwise displaced (for example having a different translation frequency) slightly relative to the original set. The overlapping sets of lines produce certain identifiable irregularities and interference patterns in the position of the gross-scale light-dark portions of the pattern 210 (see for example, FIGS. 5-6). These irregular light-dark patterns may form, in effect, a gross-scale “barcode” in which certain information can be encoded.

As shown in FIG. 5, which is a schematic representation of the moiré pattern 210 according to one example, the moiré pattern 210 can be a single repeating pattern formed from a first set of lines 212 that overlaps at least one second set 214 of lines. In one embodiment, two horizontal darker portions or regions are visible within the pattern 210. Alternatively according to another embodiment, as shown in FIG. 6, a more complex pattern 250 can be created by including a number of sections 216 with parallel lines arranged with subtly different spacing and/or angles, for example along the length of the lines. According to this example, when one or more overlapping sets of parallel lines at a different orientation are placed over the sections 216, multiple unique darker horizontal regions and patterns are created. These more complex patterns are harder to copy and, as such, can offer increased protection against counterfeiting or mislabeling of syringes, while still retaining the capability of storing identifying information regarding the syringe and/or syringe contents.

With reference again to FIGS. 4A and 4B, in certain embodiments, the moiré pattern 210 is designed to be read in an axial (i.e. vertical as shown in FIGS. 4A and 4B) direction along the syringe barrel 18. More specifically, as the syringe 12 is inserted in an injector, a vertical or axial section of the pattern 210 passes through a field of view of a scanner or reader to detect all of the information encoded within the pattern 210. For example, the moiré pattern 210 may extend around a portion of a proximal portion of syringe 12, and in certain embodiments may extend around an entire circumference of a proximal portion of syringe 12. Thus, in this example, the user may not be required to rotate or orient the syringe 12 to expose other portions of the pattern 210 to the field of view of at least one sensor, such as the scanner or reader. The relevant information can be repeated multiple times around the circumference of the syringe barrel 18 to ensure that the pattern 210 is readable regardless of the orientation of the syringe 12 relative to the at least one sensor.

The lines can be formed on the syringe barrel 18 by etching, laser etching, scratching, or a printing technique. In other embodiments, the lines can be molded on the syringe barrel 18 surface during manufacturing, for example, by using a feature within the mold surface. The combination of lines with small alignment changes leads to a large number of possible intricate patterns. Identifying information about the syringe 12 can be associated with one or more specific patterns or one or more alignments associated with the sets of lines.

According to one example, the syringe barrel 18 includes only a single set of parallel lines. However, when viewed through the translucent or transparent syringe barrel 18, overlapping lines that produce a moiré pattern appear since lines on the wall one side of the barrel 18 will overlap lines on the wall of the opposite side of the barrel 18. In this manner, a visual moiré pattern 210 is formed from a single set of parallel lines when viewed through across the diameter of the barrel. According to certain other embodiments, the moiré pattern 210 can also include additional sets of parallel lines printed on the syringe barrel 18 to produce a more intricate pattern. In addition, further intricacies within the line patterns of moiré pattern 210 can occur due to the curvature of the syringe barrel 18.

The various embodiments of the moiré pattern 210 are read or captured using at least one reader or sensor, located, for example, on an inner surface of the syringe port 16. The reader or sensor may illuminate the moiré pattern with collimated light, such as light provided by a packaged light emitting diode (LED). Thus, the moiré pattern 210 can be identified and read to provide information on the syringe parameters, the injection parameters, and/or the syringe contents to the injector or operating system as described herein. According to other examples, the one or more patterns of parallel lines can be placed on one or more surface of a flange around the circumference of syringe body 18, such as drip flange 28 or locking flange 32. According to these examples, the moiré pattern 210 can form from differential overlap of a first set of parallel lines on one surface, such as a proximal surface, of the flange and at least a second set of parallel lines on an opposite surface, such as the distal surface, of the flange. In other embodiments, radial lines, extending out from the circumference of syringe barrel 18 at different angles, instead of parallel lines, can be used to form the moiré pattern 210. In other embodiments, at least one set of parallel or radial lines can be on a surface of a circumferential flange and a second set of parallel or radial lines can be arranged circumferentially around the outer surface of syringe port 16 to form a moiré pattern 210 when the syringe 12 is inserted into the syringe port 16.

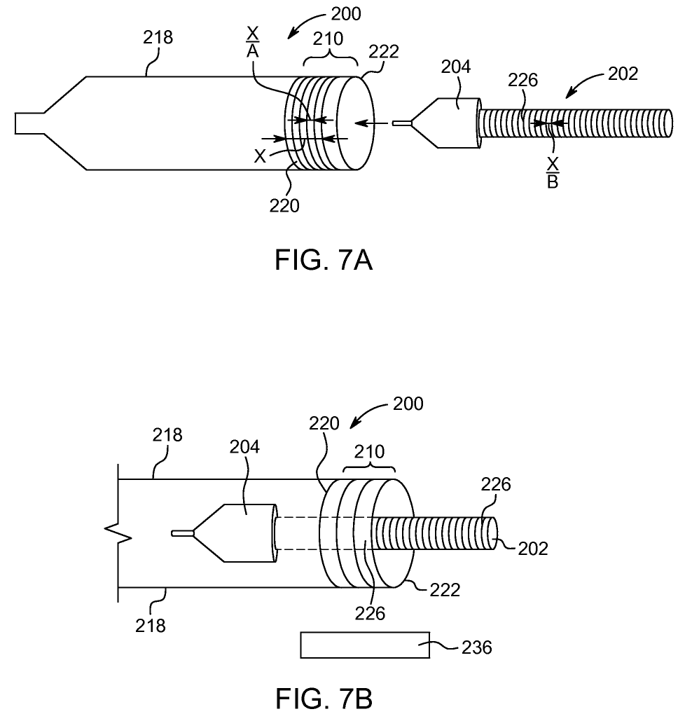

With reference to FIGS. 7A and 7B, according to another example, a syringe 200 can include a moiré pattern 210 formed from lines or rings positioned on a piston rod 202 and/or plunger 204, as well as on the syringe barrel 218. The moiré pattern 210 can be encoded or associated with identifying information about the syringe 200, injection parameters, and/or syringe contents. In this example, the otherwise translucent syringe barrel 218 includes a plurality of parallel, circumferential rings 220 or lateral lines located near the proximal end 222 of the syringe barrel 218. The parallel, circumferential rings 220 or lateral lines can be opaque or semi-opaque and can be formed as described herein. For example, the rings 220 or lines can be applied to the barrel 218 by etching, molding, laser marking, printing, or other suitable process. According to some examples, the rings 220 can be manufactured without the type of tight tolerances required by other molded or etched indicia, such as barcodes. Therefore, this embodiment can simplify the molding process and/or reduce the number of syringes that must be discarded for imperfections caused by tool wear or misalignment of the syringe barrel 218 and molding or etching means.

In one example, the rings 220 can be spaced equidistantly apart with a frequency of A/x, wherein A is a number of lines and x is an axial distance along the syringe barrel 218 over which the rings 220 are applied. The distance between adjacent lines is x/A. For example, if there are 10 lines applied over a 2 cm axial distance, the frequency A/x is 5 lines per cm and the distance x/A between adjacent lines is 0.2 cm. In this example, the piston rod 202 or outer surface of plunger 204 also includes a series of light and dark circumferential portions that form a second plurality of parallel rings 226 extending axially along the length of the rod 202 or plunger 204. The rings 226 can be formed on the piston rod 202 by etching, molding, laser marking, printing, or other suitable process. The rings 226 can be axially spaced with a frequency of B/x along the circumference of piston rod 202 or can be at a different angle than the rings 220 on the syringe barrel 218.

As shown in FIG. 7B, at least one sensor 236 or reader can be positioned in the fluid injector 10 (shown in FIG. 1) for identifying the rings 220, 226 of the syringe barrel 218 and piston rod 202, respectively, and the moiré pattern 210 formed therefrom. In some examples, the at least one sensor 236 can be associated with a source of light, such as a light emitting diode (LED) or incandescent bulb, that is reflected by the rings 220, 226 and read by the at least one sensor 236. According to another example, the sensor 236 does not need to be capable of distinguishing between the syringe rings 220 and piston rod rings 226, but is only required to determine the position of rings 220, 226 or lines within the field of view of the sensor 236 and to decode information from the resulting moiré pattern 210. According to another example, the at least one sensor 236 reads the moiré pattern 210 when the piston rod 202 is statically placed inside the syringe barrel 218 to extract information therefrom. According to another example, the sensor 236 detects a moving moiré pattern 210 that is created as the piston rod 202 is advanced through the syringe barrel 218 in the axial direction. For example, the at least one sensor 236 can be configured to determine the distance between the rings 220, 226, which dynamically changes, for example in an oscillating fashion, as the piston rod 202 is advanced through the barrel 218 of each of the at least one syringes 200. In still another embodiment, a first set of parallel lines may be placed on a transparent surface of the injector syringe port 16 that is located in front of the scan area of the at least one sensor and a second set of offset parallel lines, as described herein, may be placed on a surface of a proximal portion of the syringe 12, as described herein. Upon insertion of syringe 12 into the syringe port 16, the lines on the transparent surface and the lines on the proximal surface of the syringe overlap to form a moiré pattern that may be read by the at least one sensor. The moiré pattern will contain identifying information of the syringe 12 as described herein.

In one embodiment, at an initial position and at each periodic event thereafter, the rings 220, 226 are spaced a distance x/(B+A) apart. This specific distance can be used to identify and validate the syringe 200 for use with the specific injector. For example, the fluid injector 10 can associate a specific distance x/(B+A) with a specific size and physical dimensions of syringe 200, including, for example, syringe information associated with date of manufacture, lot number and source, barrel/plunger friction characteristics, pressure limitations, maximum or minimum flow rates, and/or fluid type, as described herein. Similarly, the fluid injector 10 can be configured to only perform an injection for a syringe 200 and piston rod 202 having a correct predetermined distance between rings. According to some examples, the distance required between rings 220, 226 can be changed and updated in the injector, as desired, after specific times, for example, by an operator or technician or by an automatic upload (when the injector assembly is in communication with a network connection). This can allow for updating of various syringe and injection parameters or account for new injection protocols or syringe designs, etc.

With continued reference to FIGS. 7A and 7B, the specific distance and/or moiré pattern formed by rings 220, 226 can also be used to determine the position of the piston rod 202 and plunger 26 within the syringe barrel 218, thereby allowing the injector to determine parameters, such as volume of fluid injected, volume of fluid remaining, and/or rate of injection. In a specific example, the piston rod 202 travels through the barrel a distance x/A in each period, depending on the rate of injection. Therefore, each time that the specific distance x/(B+A) is identified by the sensor 236, for example by reading specific repeating pattern occurrences in the moiré pattern, the piston rod 202 has traveled another period distance x/A. The sensor 236 and controller 15 (shown in FIG. 1) of the injector 10 can be configured to determine the number of periods and, based on that value, determine the distance traveled by the piston rod 202 and plunger 204 through the syringe barrel 218, as well as the rate of travel, for example by measuring the time between the periodic pattern occurrences. This information is used for real-time monitoring of an injection procedure and/or to confirm that the injection was performed (i.e., the desired amount of medical fluid and/or has been injected). The information can also be used to determine parameters including the fluid volume injected to the patient, fluid flow rate, and/or injection pressure or force. This information about the injection, along with other data such as the time that the injection was performed, name of the technician or operator, or other relevant data, could also be electronically stored by the controller 15 in a patient record that can later be saved, for example, by uploading to a hospital or clinic network and made available for physician review or during future injections.

Although the disclosure has been described in detail for the purpose of illustration based on what is currently considered to be the most practical and preferred embodiments, it is to be understood that such detail is solely for that purpose and that the disclosure is not limited to the disclosed embodiments, but, on the contrary, is intended to cover modifications and equivalent arrangements. For example, it is to be understood that the present disclosure contemplates that, to the extent possible, one or more features of any embodiment can be combined with one or more features of any other embodiment.

1. A syringe comprising:
a syringe barrel comprising a proximal end, a distal end, and a sidewall extending therebetween; and
at least one indicia located on a portion of the syringe barrel containing or associated with identifying information about the syringe, one or more injection parameters of a fluid injector, or a fluid filled in the syringe barrel,
wherein the at least one indicia is readable by a reader independent of an orientation of the syringe relative to the reader.

2. The syringe of claim 1, wherein the at least one indicia comprises one or more markings etched into a portion of the syringe barrel.

3. The syringe of claim 1, wherein the at least one indicia comprises one or more computer readable barcodes.

4. The syringe of claim 1, wherein the at least one indicia comprises a plurality of repeating barcode sections.

5. The syringe of claim 4, wherein each of the plurality of repeating barcode section comprises all of the identifying information of the at least one indicia.

6. (canceled)

7. The syringe of claim 1, wherein the at least one indicia is located on a portion of the syringe barrel configured to be inserted into a syringe port of a fluid injector.

8. The syringe of claim 1, wherein the at least one indicia is located on a proximal surface or a distal surface of an annular flange extending from the syringe barrel.

9. The syringe of claim 1, wherein the at least one indicia comprises a first set of parallel lines at least partially overlapping with a second set of parallel lines having a different orientation from the first set of parallel lines, thereby forming a moiré pattern.

10. The syringe of claim 9, wherein the first set of parallel lines and the second set of parallel lines are each located on the syringe barrel.

11. The syringe of claim 9, wherein the syringe further comprises a plunger and a piston rod, and wherein the second set of parallel lines is located on at least one of the piston rod and the plunger.

12. A fluid injection system comprising:
at least one syringe comprising a syringe barrel having a proximal end, a distal end, and a sidewall extending therebetween;
at least one indicia on at least a portion of the syringe barrel;
a fluid injector comprising at least one syringe receiving port configured to receive the at least one syringe;
at least one sensor positioned to obtain an image of the at least one indicia; and
a controller configured to:
obtain the image from the at least one sensor,
read the image to extract identifying information about the at least one syringe, one or more injection parameters, or information for a fluid contained or to be contained in the at least one syringe, and
provide instructions for performing an injection using the fluid injector, the instructions being based, at least in part, on the information extracted by the controller,
wherein the at least one indicia is orientation independent, and can be read by the sensor in any position relative to the syringe receiving port.

13. The fluid injection system of claim 12, wherein the at least one indicia comprises a plurality of repeating barcode sections arranged around a circumference of the syringe barrel.

14. The fluid injection system of claim 13, wherein each of the plurality of repeating barcode sections comprises four or more location identifying markers, and wherein the at least one sensor comprises a field of view having an area large enough to capture an image including four or more location identifying markers from either one barcode section or from one or more adjacent repeating barcode section, regardless of the orientation of the syringe relative to the injector port.

15. The fluid injection system of claim 14, wherein a side length of the field of view of the at least one sensor is at least \sqrt{2} times the length of a side length of one of the plurality of repeating barcode sections.

16. The fluid injection system of claim 12, wherein reading the image of the at least one indicia comprises applying an image processing algorithm to translate one or more portions of the image to produce a correctly oriented barcode section.

17. The fluid injection system of claim 12, wherein reading the image of the at least one indicia comprises applying an image processing algorithm to account for curvature of the syringe barrel.

18. (canceled)

19. The fluid injection system of claim 12, wherein the at least one indicia comprises a first set of parallel lines at least partially overlapping with a second set of parallel lines having a different orientation from the first set to form a moiré pattern.

20. The fluid injection system of claim 19, wherein the first set of parallel lines is located on the syringe barrel, and the second set of parallel lines is located on at least one of a piston rod of the fluid injector and a plunger of the at least one syringe.

21. The fluid injection system of claim 19, wherein reading the image of the at least one indicia comprises identifying a spatial relationship between the first set of parallel lines and the second set of parallel lines.

22. The fluid injection system of claim 21, wherein the controller is further configured to identify an injection rate based on the spatial relationship between the first set of parallel lines and the second set of parallel lines.
